(19) United States
(12) Patent Application Publication
(10) Pub. No.: US 2024/0024118 A1
(43) Pub. Date: Jan. 25, 2024

(54) A Mechanically Secured Plastic Patellar Prosthesis

(71) Applicant: BRANDON LEE THOMPSON, PEARLAND, TX (US)

(72) Inventor: BRANDON LEE THOMPSON, PEARLAND, TX (US)

(21) Appl. No.: 18/167,296

(22) Filed: Feb. 10, 2023

Related U.S. Application Data
(60) Provisional application No. 63/369,140, filed on Jul. 22, 2022.

(51) Int. Cl. A61F 2/38; A61B 17/17

(52) U.S. Cl. CPC A61F 2/3877; A61B 17/1767; A61F 2/3859

(57) ABSTRACT

A plastic patellar prosthesis having a plurality of orienting pins, each pin having one or more projections that define a maximum diameter greater than the diameter of the patellar hole into which the orienting pin is inserted. The diametrical interference causes the projections to deform and thereby mechanically secure the patellar prosthesis to the resurfaced patella as the cement cures without the need for a patellar clamp to hold the prosthesis in place.

### CROSS REFERENCE TO RELATED APPLICATIONS

This application for patent claims benefit of and priority to U.S. Application filed on Jul. 22, 2022, having Ser. No. 63/369,140, the contents of which are incorporated herein by reference for all purposes.

### STATEMENT REGARDING FEDERALLY SPONSORED RESEARCH OR DEVELOPMENT

Not applicable.

### REFERENCE TO APPENDIX

Not applicable.

### BACKGROUND OF THE INVENTION

### Field of the Invention

The inventions disclosed herein relate to plastic patellar prostheses used during total knee arthroplasty in which the patellar prosthesis is cemented is to the resurfaced patella.

### Description of the Related Art

Total knee arthroplasty involves three components: a femoral component, a tibial component, and a patellar component and numerous surgical tools. For the patellar component, the posterior surface of the existing patella (i.e., the articular side; the side that previously engaged the natural femoral condyles) is resurfaced, such as by sawing, to reduce the thickness of the existing patella and to form a typically planar surface. A patellar prothesis is provided for the total knee replacement and has a posterior or articular surface designed to operatively engage with the prosthetic “condyles” and an anterior surface designed to mate with the resurfaced portion of the existing patella.

Conventional patellar prostheses typically have orienting lugs that protrude from the anterior surface and engage corresponding receptacles formed in the resurfaced patella. These conventional lugs and receptacles orient the patellar prosthesis relative to the patella and femoral component. The plastic prosthesis is secured to the existing patella typically by bone cement or for metal-backed protheses, by an interference or press fit between the lugs and receptacles.

Conventional plastic patellar prostheses are secured to the patella with adhesive, such as bone cement. Because bone cement requires time to cure or set (for example, 20 minutes) a patellar clamp is used to apply a compressive force to the patellar prothesis/patella combination while the bone cement sets. While the bone cement is setting (and the patellar clamp is applied), the knee replacement surgery is typically interrupted.

For examples, U.S. Pat. No. 5,871,540 discloses conventional plastic patellar prostheses in FIGS. 5-10.

U.S. Pat. No. 10,034,774 discloses a Disposable Modular Patella is Protector/Clamp.

The present inventions are directed to improved plastic patellar prostheses that do not require a patellar clamp while the bone cement hardens or cures.

### BRIEF SUMMARY OF THE INVENTION

Brief, non-limiting summaries of my inventions as presently claimed are:

A patellar prosthesis may comprise an anterior surface adapted to mate with a corresponding prepared surface of a natural patella, the anterior surface may have a plurality of pins extending from the anterior surface, wherein at least one of the plurality of pins can be formed from a plastic material, and, wherein each of the plurality of pins has an outer surface between an end and the anterior surface. The one or more projections on the at least one plastic pin may extend outwardly from the outer surface of the at least one plastic pin. The one or more projections on the at least one plastic pin may contact a surface of a hole formed in the prepared surface of the patella into which the pin is inserted and thereby mechanically restrain the patellar prothesis from moving relative to the natural patella while bone cement cures between the anterior surface and the prepared surface of the natural patella.

A patellar prosthesis may be formed from a medically safe plastic material. The plurality of pins may be integral to the patellar prosthesis. The plurality of pins may have one or more projections that are integral with the pins. The one or more projections may extend along at least a portion of the length of the pin. The one or more projections may extend along the length of the pins. The patellar prosthesis may have three pins. Each pin may have three equally spaced projections. Each projection may extend axially along at least a portion of the length of the pin. The anterior surface may further comprise a planar surface and at least one recess in the planar surface. The three pins may emanate from the planar surface. The outer surface of each pin may comprise one or more recesses. The anterior surface may further comprise a planar surface and at least one recess in the planar is surface. The three pins may emanate from the planar surface. The outer surface of each pin may comprise one or more recesses. The one or more projections may have a serrated edge for engaging the hole surface. One or more of the one or more projections may be oriented circumferentially on at least one of the plurality of pins. One or more of the projections may be angled relative to a longitudinal pin axis on at least one of the plurality of pins. Each of the plurality of pins may comprise a circumferential projection and at least one non-circumferential projection.

A total knee prosthesis may compromise a femoral component, a tibial component, and a patellar component integrally fabricated from a medically safe plastic. The patellar component may further comprise a posterior surface shaped to operatively engage the femoral component, an anterior surface configured for mating with a resurfaced portion of a natural patella. The anterior surface may have a plurality of pins extending from the anterior surface, wherein each pin has an outer surface extending between a pin end and the anterior surface. A plurality of projections on each pin may extend radially outward from the outer surface of the pin. The plurality of projections on each pin may define one or more maximum diameters that are greater than a pin diameter and equal to or greater than a diameter of a hole formed in the natural patella into which the pin is pressed such that the plurality of projections mechanically secures the patellar component to the natural patella to inhibit movement while bone cement cures between the patellar component and the patella.

The plurality of pins may be three, and the plurality of projections on each pin may be three equally spaced about each pin. The maximum diameter of each pin may be greater than the diameter of a hole in the natural patella into which each pin is inserted.

None of these brief summaries of the inventions as presently claimed is intended to limit or otherwise affect the scope of what has been disclosed and enabled or the appended claims, and nothing stated in these brief summaries is intended as a definition of a claim is term or phrase or as a disavowal or disclaimer of current or future claim scope.

### BRIEF DESCRIPTION OF THE SEVERAL VIEWS OF THE DRAWINGS

The following figures form part of the disclosure of inventions and are included to demonstrate further certain aspects of the inventions. The inventions may be better understood by reference to one or more of these figures in combination with the detailed description of certain embodiments presented herein.

FIG. 1 illustrates the components of a human knee.

FIG. 2 illustrates a conventional artificial knee utilizing a patellar prothesis.

FIGS. 3A and 3B illustrate a conventional plastic patellar prothesis cemented to a resurfaced patella as part of a total knee arthroplasty.

FIGS. 4A through 4H illustrate alternate patellar prothesis pins with projections according to my invention providing a mechanical bond to a resurfaced patella.

FIG. 5 illustrates an alternate pin configuration comprising a central channel

FIG. 6 illustrates a pin with projections according to my invention placed in a resurfaced patella hole and in which the projections are deformed.

FIG. 7 illustrates a plastic patella prosthesis utilizing orientation pins with different projections.

FIGS. 8A and 8B illustrate another embodiment of a patellar prosthesis.

FIG. 9 illustrates a bottom view of one of the pins illustrated in FIGS. 8A and 8B.

FIG. 10 illustrates a side view of the pin illustrated in in FIG. 9.

While the inventions disclosed herein are susceptible to various modifications and alternative forms, only a few specific embodiments have been shown by way of example in the drawings and are described in more detail below. The figures and detailed descriptions of these embodiments are not intended to limit the breadth or scope of the inventive concepts or the appended claims in any manner Rather, the figures and detailed written descriptions are provided to illustrate the inventive concepts to a person of ordinary is skill in the art and to enable such person to make and use the inventive concepts illustrated and taught by the specific embodiments.

### DETAILED DESCRIPTION

The Figures described above, and the written description of specific structures and functions below, are not presented to limit the scope of the inventions disclosed or the scope of the appended claims. Rather, the Figures and written description are provided to teach a person skilled in this art to make and use the inventions for which patent protection is sought.

A person of skill in this art having benefit of this disclosure will understand that the inventions are disclosed and taught herein by reference to specific embodiments, and that these specific embodiments are susceptible to numerous and various modifications and alternative forms without departing from the inventions we possess. For example, and not limitation, a person of skill in this art having benefit of this disclosure will understand that Figures and/or embodiments that use one or more common structures or elements, such as a structure or an element identified by a common reference number, are linked together for all purposes of supporting and enabling our inventions, and that such individual Figures or embodiments are not disparate disclosures. A person of skill in this art having benefit of this disclosure immediately will recognize and understand the various other embodiments of our inventions having one or more of the structures or elements illustrated and/or described in the various linked embodiments. In other words, not all possible embodiments of our inventions are described or illustrated in this application, and one or more of the claims to our inventions may not be directed to a specific, disclosed example. Nonetheless, a person of skill in this art having benefited from this disclosure will understand that the claims are fully supported by the entirety of this disclosure.

People skilled in this art will appreciate that not all features of a commercial is embodiment of the inventions are described or shown for the sake of clarity and understanding. Persons of skill in this art will also appreciate that the development of an actual commercial embodiment incorporating aspects of the present inventions will require numerous implementation-specific decisions to achieve the developer's ultimate goal for the commercial embodiment. Such implementation-specific decisions may include, and likely are not limited to, compliance with system-related, business-related, government-related, and other constraints, which may vary by specific implementation, location and from time to time. While a developer's efforts might be complex and time-consuming in an absolute sense, such efforts would be, nevertheless, a routine undertaking for those of skill in this art having benefit of this disclosure.

Further, the use of a singular term, such as, but not limited to, “a,” is not intended

as limiting of the number of items. Also, the use of relational terms, such as, but not limited to, “top,” “bottom,” “left,” “right,” “upper,” “lower,” “down,” “up,” “side,” and the like are used in the written description for clarity in specific reference to the Figures and are not intended to limit the scope of the invention or the scope of what is claimed.

Reference throughout this disclosure to “one embodiment,” “an embodiment,” or similar language means that a particular feature, structure, or characteristic described in connection with the embodiment is included in at least one of the many possible embodiments of the present inventions. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

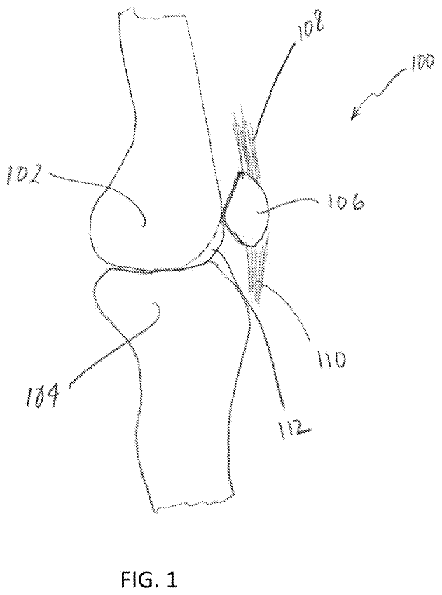

FIG. 1 illustrates the anatomy of a human knee 100 showing the femur 102, the tibia 104, the patella 106 and the patellar tendons 108 and 110. The trochlear groove 112, in which the posterior or articular side of patella slides, is also shown.

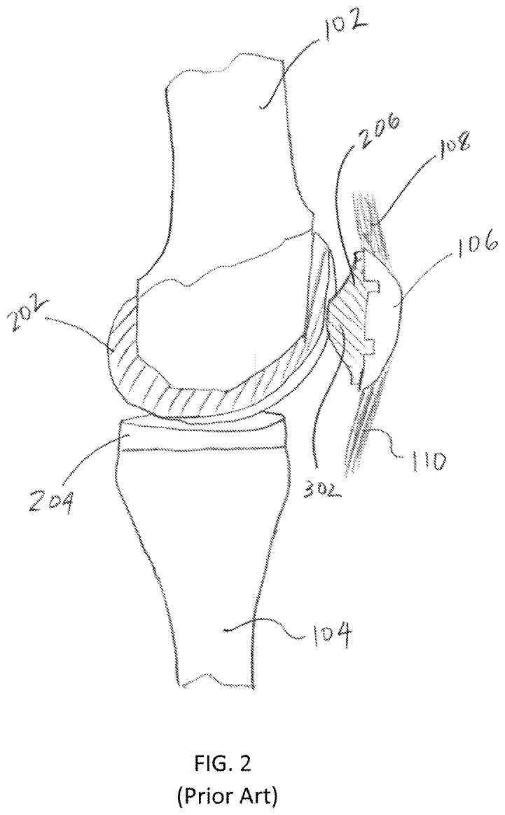

FIG. 2 illustrates a conventional total knee arthroplasty or knee replacement 200 on is a human knee. The knee prosthesis includes a femoral component 202, a tibial component 204, and a patellar component 206. The patellar component 206 is shown coupled to the resurfaced portion of the existing patella 106. In this illustration, the patellar component 206 is plastic and is cemented to the resurfaced patella 106. As discussed above, to prevent the patellar component 206 from loosening or displacing from the patella 106 while the cement cures, a patellar clamp (not shown) is used to hold the patellar component 206 in position until the cement hardens or cures sufficiently that the clamp can be removed, and the replacement surgery can proceed.

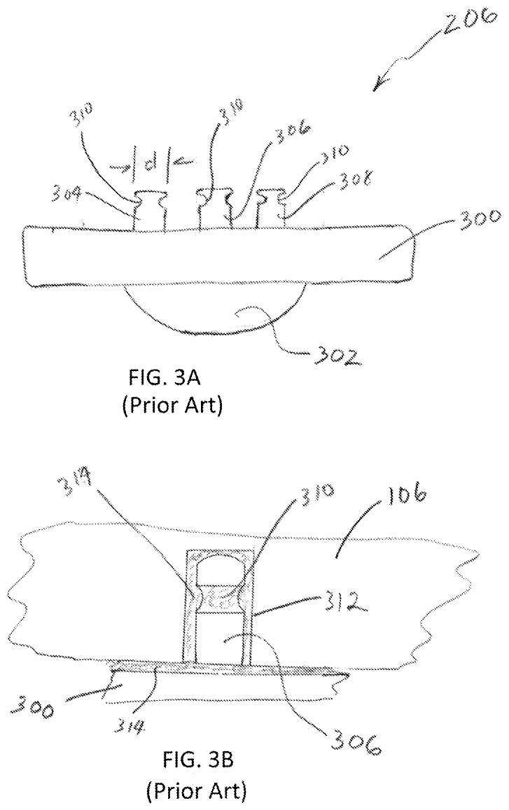

FIG. 3A illustrates a side view of the conventional plastic patellar component 206 illustrated in FIG. 2. The patellar component 206 comprises a base 300 and an articular surface 302. Opposite from the articular surface 302, the component 206 has three cylindrical pins 304, 306 and 308 extending away from the base 300. Each pin has a circumferential groove 310. Typically, the three pins 306, 308, 310 are arranged at the points of an equilateral triangle.

FIG. 3B illustrates a cross-section of the patellar component 206 cemented to the patella 106. Pin 310 is shown positioned in hole 312 formed in the resurfaced portion of the patella 106. As can be seen, the pin 310 has an outer diameter that is less than the diameter of the hole 312. For example, for a hole having a diameter of 6.35 mm, the pin may have a diameter of 5.75 mm creating an annulus into which cement 314 can reside. The groove 310 provides additional surface area for cement bonding.

With the foregoing as background, I have invented plastic patellar prostheses that are mechanically secured to the patella while the bone cement between the patellar prosthesis and the resurfaced patella sets without the need for a patellar clamp or other device that interrupts the surgical procedure. Embodiments of my invention may comprise two or more orienting pins extending from an anterior surface of the prosthesis, wherein each pin comprises one or more projections emanating from the side or sides of the pin. is The projections are structurally configured, that is, sized, shaped, and/or located, so that the projections deform when the pins are inserted into the corresponding receptacles formed in the resurfaced patella. The projections mechanically secure the prosthesis to the patella, such as through an interference fit with the wall or walls of the receptacle. The projections secure the prosthesis to the patella while the bone cement cures and allows the total knee replacement surgery to proceed because a patellar clamp or other bonding tool is not needed and, therefore, not in the way. In addition to securing the prosthesis while the bone cement cures, the deformed projections provide additional mechanical security after the cement has hardened.

It is preferred, but not required, that the patellar prothesis is a unitary or integral component and not an assembly of discrete components. For example, and not limitation, a preferred embodiment may be integrally formed from a block of plastic, such as, but not limited to, polyethylene, and may comprise an anterior surface with two or more orienting pins with a plurality of projections emanating from the pins. The projections may be longitudinal with respect to the pins, circumferential with respect to the pins, angled with respect to the pins or any combinations of the foregoing. Regardless of the type of projections employed, the outer surface of the projections defines a maximum diameter, D_{M}

The pin receptacles formed in the patella, which are preferably round, may be sized with a diameter, D_{P}, which is equal to or less than the maximum projection diameter, D_{M}, so that the projections contact or deform, such as by compressing, bending, folding, or reducing, when the pins to enter the receptacles. This contact or deformation, which may be plastic (i.e., non-recoverable) or elastic (i.e., recoverable), provides a securing force sufficient to hold the prosthesis against the patella as the cement there between cures. In other words, the projections hold the prosthesis in place and overcome any hydrostatic or other forces that tend to cause the prosthesis to dislocate while the cement hardens.

Turning now to descriptions of several embodiments of my invention with reference to specific figures, persons of skill having benefitted from this disclosure will understand that the foregoing description of my invention applies to each of these specific embodiments and to those many other embodiments enabled by this description.

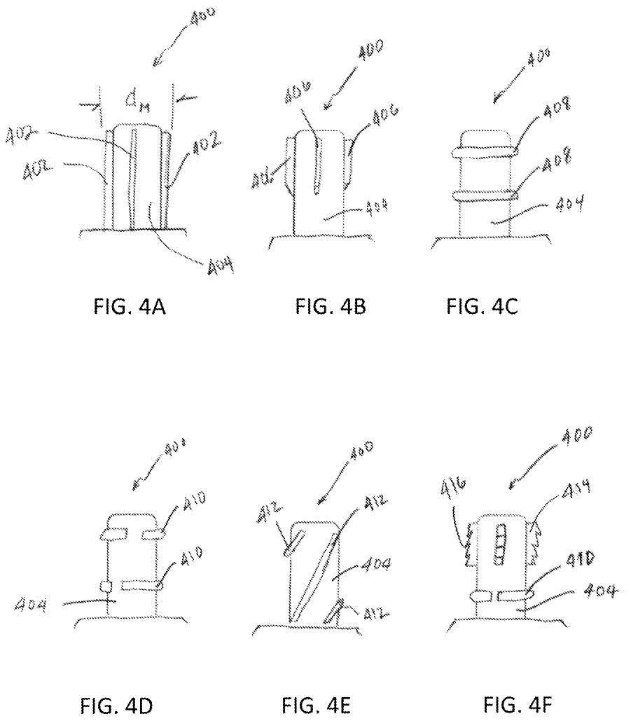

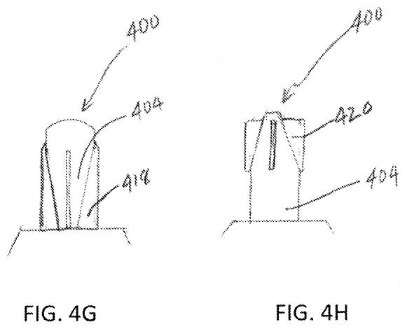

FIGS. 4A-4H illustrate individual pins 400 for use with a plastic patellar prothesis, having two or more orienting pins 400. While two pins will produce the required orienting function, three or four pins are preferred because of the additional mechanical securing force provided. While four pins may provide more mechanical securing force, the additional hole in the patella may comprise the structural integrity of the resurfaced patella. This result may be overcome by reducing the diameter of the holes (and pins). Also, my invention contemplates having pins of different sizes (diameters and/or lengths) on a single patellar prothesis.

FIG. 4A illustrates a pin 400 having a plurality of projections 402 that extend longitudinally along the outer surface 404 of the pin 400 for its length. In this embodiment the projections 402 are formed integrally with the pin 400, such as by machining or casting. The pin 400 may have an outer surface 404 diameter of 5 mm and each projection 402 may have a radial depth of 1 mm. With those dimensions, the projections 402 define a maximum undeformed diameter, d_{M}, for pin 400 of 7 mm.

FIG. 4B illustrates an alternate pin 400 having a plurality of projections 406 that extend longitudinally along the outer surface 404 of the pin 400 for less than the pin length. In this embodiment the projections 406 are formed integrally with the pin 400, such as by machining or casting. For example, if the pin 400 has a length of 5 mm, the projections 406 may have length of 4 mm. The projections 406 define a maximum undeformed diameter, dₘ, such as described for the embodiment in FIG. 4A.

FIG. 4C illustrates an alternate pin 400 having a plurality of projections 408 that extend circumferentially about the outer surface 404 of the pin 400. In this embodiment the projections 408 are formed integrally with the pin 400, such as by machining or casting. The projections 408 define a maximum undeformed diameter, d_{M}, such as described for is the embodiment in FIGS. 4A and 4B.

FIG. 4D illustrates an alternate pin 400 having a plurality of projections 410 that extend circumferentially and discontinuously about the outer surface 404 of the pin 400. In this embodiment the projections 410 are formed integrally with the pin 400, such as by machining or casting. The projections 410 define a maximum undeformed diameter, d_{M}, such as described for the embodiment in FIGS. 4A, 4B, and 4C. The discontinuous nature of the projections 410 allow cement to be extruded through the annulus formed with hole in the patella (not shown) as the pins are pushed into the holes.

FIG. 4E illustrates an alternate pin 400 having a plurality of projections 412 that are angled about the outer surface 404 of the pin 400 with respect to the longitudinal axis of the pin. In this embodiment the projections 412 are formed integrally with the pin 400, such as by machining or casting. The projections 412 define a maximum undeformed diameter, d_{M}, such as described for the embodiment in FIGS. 4A, 4B, 4C, and 4D. It is contemplated that the projections 412 may extend the length of the pin 400, as do the projections 402 in FIG. 4A or may extend less than the pin 400 length as illustrated in FIG. 4B.

FIG. 4F illustrates an alternate pin 400 having a plurality of projections that have jagged or serrated outer edge 416. As illustrated, the serrations 416 are oriented to engage the hole wall and help prevent the pin (and therefore the prothesis) from dislocating from the patella (not shown). Although the serrations are illustrated on the projections illustrated in FIG. 4B, it is to be understood that serrated edges can be utilized with the projections illustrated or described herein, including projections 408 and 410.

FIG. 4F also illustrates that a single pin may employ different types of projections. For example, and not limitation, the alternate pin 400 of FIG. 4F comprises a plurality of longitudinal projections 414 that do not extend for the entire pin length and discontinuous circumferential projections 410.

While the orienting pins 400 in FIGS. 4A through 4H are illustrated as cylindrical, it is will be appreciated that other pin shapes may be utilized. For example, FIGS. 4G and 4H illustrate pyramidal pins with longitudinal projections 418 and 420. It will be appreciated that any of the projections disclosed herein may be utilized with these non-cylindrical orienting pins.

It is also contemplated that a patellar prothesis may comprise different pins on the prothesis. For example, and not limitation, a plastic patellar prothesis according to my inventions may comprise a first pin 400 as illustrated in FIG. 4A, a second pin 400 illustrated in FIG. 4E and a third pin 400 as illustrated in FIG. 4E or 4H. Some or all of the projections may have serrated edges.

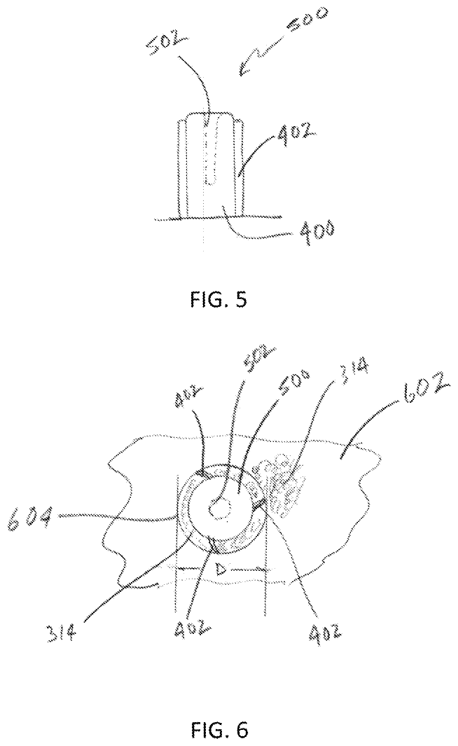

FIG. 5 illustrates an alternate pin 500, which comprises a central channel 502 along with projections 402. It will be understood that the projections may comprise any of the projections previously described. The central channel 502 may provide a space for extruded cement to locate as the pin 500 is pressed into the patellar hole (not shown).

FIG. 6 illustrates a portion of a patella 602 that has been resurfaced during a total knee arthroplasty and into which a hole 604 has been drilled to anchor a patellar prothesis (not shown). Shown inside the hole 604 is illustrated a pin 500 having three projections 402 and a channel 502. In this illustration, the maximum projection diameter, d_{M}, is larger than the diameter, D, of the hole 604. As can be seen in FIG. 6, the projections 402 are deformed because of the diametrical interference and this deformation secures the position of the patellar prothesis to the patella while the cement 314 hardens.

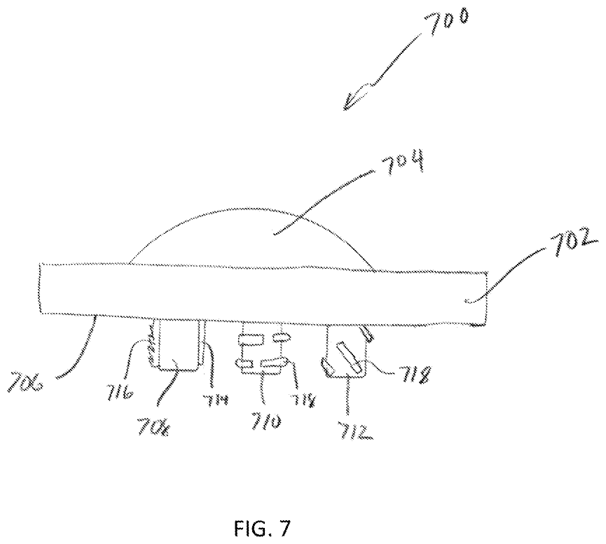

FIG. 7 illustrates a patellar prosthesis utilizing several aspects of my invention. The prosthesis 700 is fabricated as a unitary structure from implantable grade plastic, such as polyethylene, and comprises a base 702, an articular surface 704 structurally configured to operatively engage with at least the femoral component of the prosthetic knee. On the side of the base opposite the articular surface 704 are three orientation pins 708, 710, and 712. is In this illustration, each of the orientation pins have different projections, but it will be understood that one or more of the pins, including all may have the same projections. For completeness, pin 708 may comprise one or more longitudinal projections 714 and/or 716. Projection 716 comprises serrations oriented to resist axial movement out of its hole in the patella. Pin 710 comprises discontinuous circumferential projections 718. Pin 712 comprises angled projections having a length less than the pin length.

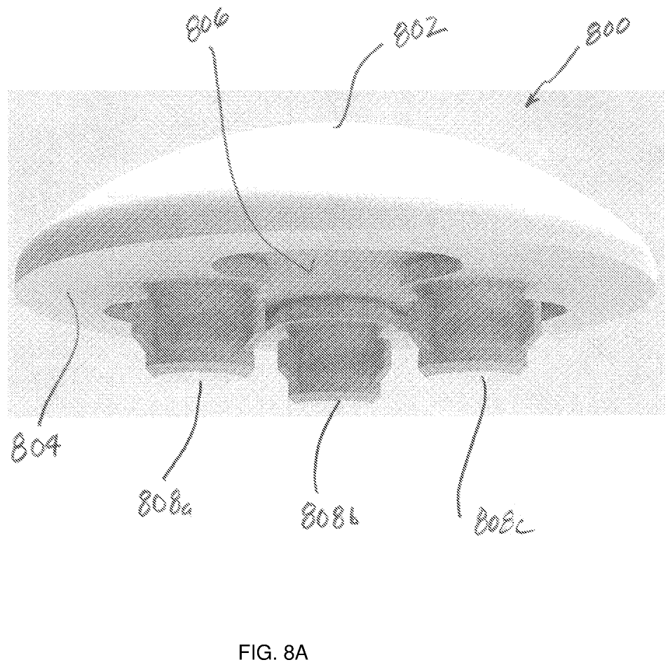

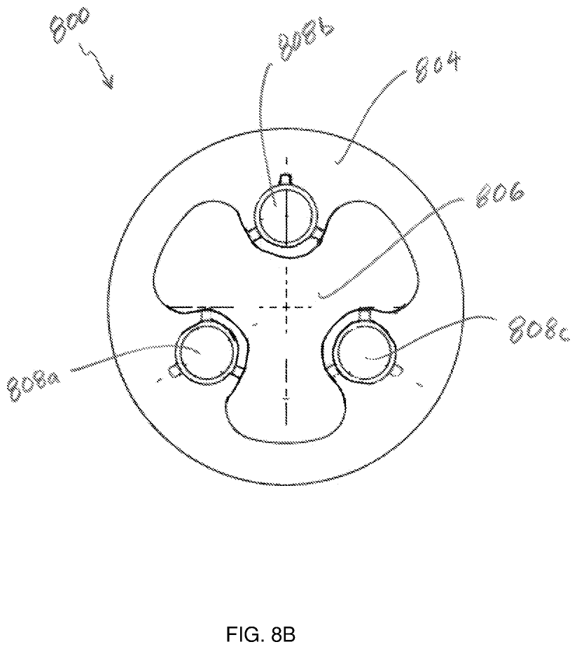

FIG. 8A illustrates a perspective view of a presently preferred embodiment of patellar prosthesis 800 of the many possible embodiments enabled by this disclosure. Prothesis 800 may be integrally fabricated from ultra-high molecular weight polyethylene (UHMWPE) and have an articular surface 802 optimized for the knee under reconstruction. The planar surface 804 is structurally configured to engage the prepared or resurfaced portion of the native patella (not shown). The planar surface 804 may comprise a single recessed 806 or multiple recesses, any of which increase the surface area for the bone cement to adhere to.

As illustrated in FIG. 8A, the prosthesis 800 may have three anchoring/alignment pins 808a, 808b and 808c. It is preferred, but not required, that the pins emanate from planar surface 804 and not from the recessed or recesses 806, but one or all of the pins may emanate from the recessed surface.

FIG. 8B illustrates a view of the anterior surface of the prothesis 800. This view illustrates that each pin 808 emanates from the planar surface 804 and each pin 808 has three projections equally spaced about the outer surface of the pin. While this embodiment has the three pins oriented at the vertices of an equilateral triangle, those of skill will appreciate that other pin orientation are contemplated within this disclosure.

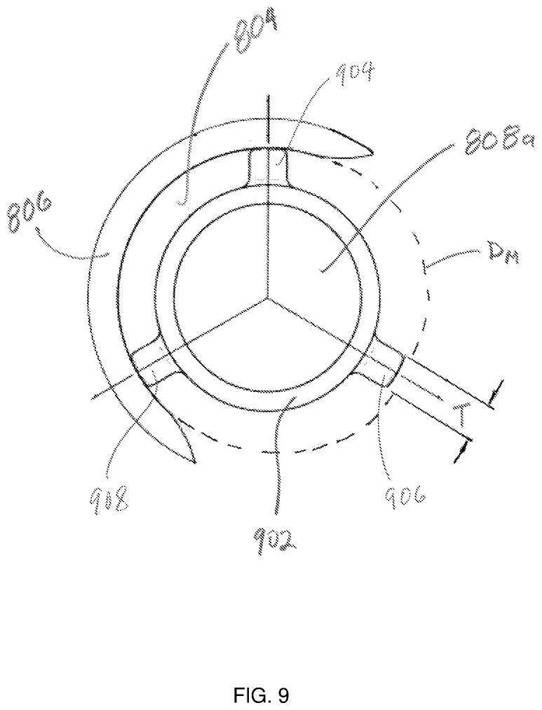

FIG. 9 illustrates pin 808a shown in FIGS. 8A and 8B. A portion of the planar surface 804 from which the pin 808a emanates is seen, as well as a portion of the recess 806. As illustrated in FIGS. 8 and 9, pin 808a preferably has a chamfered surface 902 at its distal is end to aid insertion of the pin 808a into the corresponding hole that has been drilled or reamed into the resurfaced patella.

Pin 808a may have three projections 904, 906 and 908 extending radially from the outer surface 910 of the pin 808a. The three projections are preferably spaced symmetrically on the pin, 120 degrees apart. For this particular prosthesis 800, the projections 904, 906 and 908 may have a projection thickness, T, of about 1 mm.

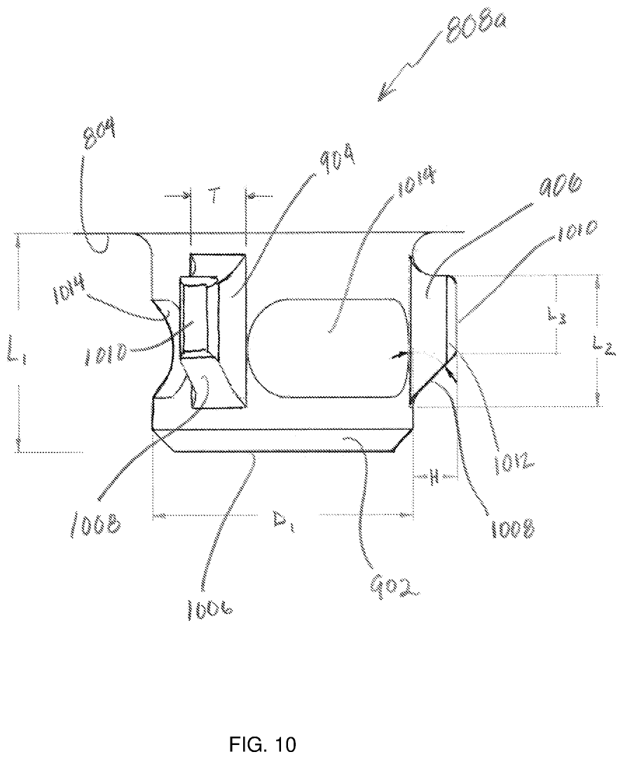

FIG. 10 illustrates a side view of the patellar prothesis pin 808a and shows two of the three projections 904 and 906. The pin body 904 diameter, D₁ is preferably 5 mm, but may range from about 3 mm to about 7 or 8 mm, depending on, among other things, the number of pins employed and the size of the patellar prosthesis. Because of chamfer 902, the distal end 1006 of the pin may have a diameter less than D₁. The pin 808a may have an axial length, L₁, from the planar surface 804 of about 5 mm, but may range from about 3 mm to about 7 or 8 mm. It is contemplated that the pin can be square in dimensions (i.e., the diameter, D₁, and the length, L₁, are substantially equal) or the pin can be elongated or truncated in the length dimension.

FIG. 10 illustrates that the projections 904, 906 and 908 (not shown) may have an overall length or base length, L₂, of about 3 mm, but may range between about 100% to about 20% of the length, L₁. The particular projections illustrated in FIGS. 8A, 8B, 9 and have a tapered surface 1008, which creates, for this embodiment, an external surface length L₃ of about 2 mm. For this embodiment, the surface 1008 is angled 45 degrees with respect to a longitudinal axis of the pin. The outer surface 1010 of the projections 904, 906 and 908 (not shown) may have chamfers 1012.

FIGS. 8A, 8B, 9 and 10 illustrates that the pins, such as 808a, may comprise channels 1014 and/or grooves formed in the pin to provide additional surface area for cement bonding.

With the foregoing disclosure, those of skill in this art are enabled to envision and is fabricate plastic patellar protheses having a plurality of orientation pins for engagement with corresponding holes formed in the resurfaced patella. One, some or all of the pins may have projections that define a maximum diameter equal to or greater than the diameter of the hole into which pin will reside. For avoidance of doubt, patellar protheses utilizing my inventions may have, for example, one pin fashioned like pin 808a, another pin fashioned like a conventional pin. Alternately, patellar protheses utilizing my inventions may have, for example, one pin fashioned like pin 808a, and one or more pins fashioned as disclosed herein in FIGS. 4A-10. Persons of skill having this disclosure will be able to fashion patellar prostheses having one or more pins that create a mechanical lock or bond at least while the cement hardens or cures without the need for a patella clamp or other tool to facilitate bonding.

After bone cement has been applied to the patellar prothesis and/or resurfaced patella, the patellar prothesis is pressed on to the resurfaced and drilled patella, such as by hand or clamping tool so the pins with projections are pressed into the corresponding holes thereby deforming the projections because of the diametrical interference. If used, the patellar clamp can be immediately removed as the projections anchor the patellar prothesis to the patella and hold the prosthesis in position as the cement hardens without the need for a patellar clamp remaining thereon.

My invention has the benefits of reducing the time needed for a total knee arthroplasty because the surgery may proceed while the patellar cement hardens, and/or providing additional mechanical security for the cemented patellar prothesis, and/or reduces the number of tools needed for arthroplasty, and/or reduces the cost of arthroplasty.

Other and further embodiments utilizing one or more aspects of the inventions described above can be devised without departing from the spirit of my invention. Further, the various methods and embodiments of the methods of manufacture and assembly of the system, as well as location specifications, can be included in combination with each other to produce variations of the disclosed methods and embodiments. Discussion of singular is elements can include plural elements and vice versa.

The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components having multiple functions.

The inventions have been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by me, but rather, in conformity with the patent laws, I intend to protect fully all such modifications and improvements that come within the scope or range of equivalent of the following claims.

What is claimed:

1. A patellar prosthesis, comprising:
an anterior surface adapted to mate with a corresponding prepared surface of a natural patella;
the anterior surface having a plurality of pins extending from the anterior surface, wherein at least one of the plurality of pins is formed from a plastic material, and, wherein each of the plurality of pins has an outer surface between an end and the anterior surface;
one or more projections on the at least one plastic pin extending outwardly from the outer surface of the at least one plastic pin; and
wherein the one or more projections on the at least one plastic pin contacts a surface of a hole formed in the prepared surface of the patella into which the pin is inserted and thereby mechanically restrains the patellar prothesis from moving relative to the natural patella while bone cement cures between the anterior surface and the is prepared surface of the natural patella.

2. The patellar prosthesis of claim 1, wherein the patellar prosthesis is formed from a medically safe plastic material.

3. The patellar prosthesis of claim 2, wherein the plurality of pins are integral to the patellar prosthesis.

4. The patellar prosthesis of claim 3, wherein each of the plurality of pins has one or more projections that are integral with the pins.

5. The patellar prosthesis of claim 4, wherein the one or more projections extend along at least a portion of a length of the pin.

6. The patellar prosthesis of claim 5, wherein the one or more projections extend along the length of the pins.

7. The patellar prosthesis of claim 4, plurality of pins is three.

8. The patellar prosthesis of claim 7, wherein each pin has three equally spaced projections.

9. The patellar prosthesis of claim 8, wherein each projection extends axially along at least a portion of a length of the pin.

10. The patellar prosthesis of claim 9, wherein the anterior surface further comprises a planar surface and at least one recess in the planar surface.

11. The patellar prosthesis of claim 10, wherein the three pins emanate from the planar surface.

12. The patellar prosthesis of claim 9, wherein the outer surface of each pin comprises one or more recesses.

13. The patellar prosthesis of claim 9, wherein the anterior surface further comprises a planar surface and at least one recess in the planar surface; wherein the three pins emanate from the planar surface; and wherein the outer surface of each pin comprises one or more recesses.

14. The patellar prosthesis of claim 4, wherein the one or more projections have a serrated edge for engaging the hole surface.

15. The patellar prosthesis of claim 4, wherein one or more of the one or more projections are oriented circumferentially on at least one of the plurality of pins.

16. The patellar prosthesis of claim 4, wherein one or more of the one or more projections are angled relative to a longitudinal pin axis on at least one of the plurality of pins.

17. The patellar prosthesis of claim 4, wherein each of the plurality of pins comprise a circumferential projection and at least one non-circumferential projection.

18. A total knee prosthesis, comprising:
a femoral component;
a tibial component; and
a patellar component integrally fabricated from a medically safe plastic;
the patellar component further comprising:
a posterior surface shaped to operatively engage the femoral component an anterior surface configured for mating with a resurfaced portion of a natural patella;
the anterior surface having a plurality of pins extending from the anterior surface, wherein each pin has an outer surface extending between a pin end and the anterior surface;
a plurality of projections on each pin extending radially outward from the outer surface of the pin; and
wherein the plurality of projections on each pin define one or more maximum is diameters that are greater than a pin diameter and equal to or greater than a diameter of a hole formed in the natural patella into which the pin is pressed such that the plurality of projections mechanically secure the patellar component to the natural patella to inhibit movement while bone cement cures between the patellar component and the patella.

19. The prosthesis of claim 18, wherein the plurality of pins is three, and wherein the plurality of projections on each pin are three equally spaced about each pin.

20. The prosthesis of claim 19, wherein the maximum diameter of each pin is greater than a diameter of a hole in the natural patella into which each pin is inserted.
